(19) United States
(12) Patent Application Publication
(10) Pub. No.: US 2023/0141289 A1
(43) Pub. Date: May 11, 2023

(54) Fertility/Reproductive Health System, Protocols, And Methods For Assisting Fertility/Reproductive Health

(71) Applicant: Famlee Fertility, Inc., Portland, OR (US)

(72) Inventor: Shelley Koranda, Portland, OR (US)

(73) Assignee: Famlee Fertility, Inc., Portland, OR (US)

(21) Appl. No.: 17/983,207

(22) Filed: Nov. 8, 2022

Related U.S. Application Data
(60) Provisional application No. 63/277,409, filed on Nov. 9, 2021.

(51) Int. Cl. G16H 20/10; G16H 10/60; G16H 80/00

(52) U.S. Cl. CPC G16H 20/10; G16H 10/60; G16H 80/00

(57) ABSTRACT

A fertility/reproductive health system and methods. The fertility/reproductive health system may include a server, the server may include a fertility/reproductive health application, a controller, operating memory, and a communications interface, wherein the server may be accessible via a network. The fertility/reproductive health system may further include a data store in communication with the server; and wherein the controller is configured to execute stored program instructions, that may include providing access to a user and one or more provider entities; receiving health data of a user; processing the received health data of the user; generating notifications to and/or from one or both of the user and the one or more provider entities; and generating fertility/reproductive treatment suggestions based on the processed health data of the user.

### RELATED APPLICATIONS

This application is related and claims priority to U.S. Provisional Patent Application No. 63/277,409, filed on Nov. 9, 2021, the application of which is incorporate herein by reference in its entirety.

### TECHNICAL FIELD

The subject matter herein relates generally to fertility and/or reproductive healthcare for men and/or women and more particularly to a health system, protocols, and methods for assisting fertility/ reproductive health, which may include, but is not limited to, fertility health for, trimester-one pregnancy health, and/or postpartum health.

### BACKGROUND

One in five couples experience unexplained infertility. Currently, fertility clinics and/or reproduction healthcare providers test only for those hormones that are related specifically to fertility. The treatment protocols of these fertility clinics and/or reproduction healthcare providers may ignore the fact that fertility is a direct result of an individual's whole body health including the individual's overall hormonal health. Consequently, other hormonal imbalances not specifically related to fertility may go unidentified, which can result in about 40% of fertility-related issues. In vitro fertilization (IVF) can be an alternative to overcome fertility-related issues. However, a drawback of the IVF process is that IVF is very expensive and it may require multiple cycles. Therefore, new approaches are needed with respect to improving an individual's overall fertility/reproductive health for men and/or women, which may include, but is not limited to, fertility health, trimester-one pregnancy health, and/or postpartum health, and to avoid the expensive IVF process.

### SUMMARY

In one embodiment, a fertility/reproductive health system is provided. The fertility/reproductive health system may include a server, the server may include a fertility/reproductive health application, a controller, operating memory, and a communications interface, wherein the server may be accessible via a network. The fertility/reproductive health system may further include a data store in communication with the server; and wherein the controller is configured to execute stored program instructions, that may include providing access to a user and one or more provider entities; receiving health data of a user; processing the received health data of the user; generating notifications to and/or from one or both of the user and the one or more provider entities; and generating fertility/reproductive treatment suggestions based on the processed health data of the user. The received health data may include one or more of data input by the user, responses to fertility/reproductive health intake protocols by the user, user's lab test results, and health data of the user received from one or more provider entities. The controller may be further configured to execute stored program instructions that may include, sending one or more instructions to the user, based on at least some of the processed health data of the user, instructing the user to take certain lab tests at certain times. The one or more of the lab tests may include tests for one or more of anti-mullerian hormone (AMH), thyroid-stimulating hormone (TSH), follicle-stimulating hormone (FSH), estradiol (E2), luteinizing hormone (LH), progesterone (Pg), and total testosterone (TT), thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), and/or other hormones potentially related to fertility/reproductive health. The one or more of the lab tests may include tests for one or more of saliva testing for men or women and/or semen analysis including one or more of semen volume, concentration, count, motility, total motile count, morphology and/or DNA fragmentation. The generating of fertility/reproductive treatment suggestions may include comparing the health data of the user to a set of fertility/reproductive treatment protocols. The user may include an individual seeking treatment for a fertility/reproductive health issue. The one or more provider entities may include one or more of a healthcare provider, laboratory service provider, pharmacy, and/or an in vitro fertilization (IVF) provider. The healthcare provider may include a telehealth provider. The user may access the fertility health application of the fertility health system through the network via a web-based portal via a computing device. The user may access the fertility health application of the fertility health system via a local fertility health application installed on a computing device.

In another embodiment, a method of auser using a fertility/reproductive health system is provided. The method may include accessing a fertility/reproductive health system by a user, the fertility/reproductive health system. The fertility/reproductive health system may include a server, the server may include a fertility/reproductive health application, a controller, operating memory, and a communications interface, wherein the server may be accessible via a network. The fertility/reproductive health system may further include a data store in communication with the server; and wherein the controller is configured to execute stored program instructions, that may include providing access to a user and one or more provider entities; receiving health data of a user; processing the received health data of the user; generating notifications to and/or from one or both of the user and the one or more provider entities; and generating fertility/reproductive treatment suggestions based on the processed health data of the user. The method may further include entering health data of the user; user taking lab tests as and when instructed by the fertility/reproductive health application; entering lab test results into the fertility/reproductive health application; and user consulting with a healthcare provider. The health data of the user may include one or more of health data input directly by the user, responses to fertility/reproductive health intake protocols by the user, and/or health data of the user received from one or more provider entities. The lab test results may be entered by one or more of the user, laboratory service provider, and healthcare provider. The healthcare provider may include a telehealth provider. The method may further include receiving fertility/reproductive treatment suggestions from the healthcare provider, the fertility/reproductive treatment suggestions generated based on comparing the entered health data of the user and lab test results of the user to a set of fertility/reproductive treatment protocols stored in the data store. The lab tests may be either or both of at-home and/or at-lab lab tests. The step of the user taking lab tests may include the user, when instructed by the fertility/reproductive health application, first a day-two menstrual cycle test, wherein the day-two menstrual cycle test may include tests for one or more of anti-mullerian hormone (AMH), thyroid peroxidase antibody (TPoAB), free triiodothyronine (FT3), free thyroxine (FT4), fasting insulin (F1), hemoglobin A1C (HgbA1c), thyroid-stimulating hormone (TSH), dehydroepiandrosterone sulfate (DHEA-s), follicle-stimulating hormone (FSH), estradiol (E2), vitamin D (Vit D), luteinizing hormone (LH), and/or other hormones potentially related to fertility/reproductive health. The one or more of the lab tests may include tests for one or more of saliva testing for men or women and/or semen analysis including one or more of semen volume, concentration, count, motility, total motile count, morphology and/or DNA fragmentation. The step of the user taking lab tests further may include the user, after taking the day-two menstrual cycle test and/or when instructed by the fertility/reproductive health application, taking one or more ovulation tests, and wherein upon a positive ovulation predictor and/or when instructed by the fertility/reproductive health application taking a progesterone (Pg) test and/or a testosterone (TT) test.

In yet another embodiment, a method of using a fertility/reproductive health system is provided. The method may include providing a fertility/reproductive health system. The fertility/reproductive health system may include a server, the server may include a fertility/reproductive health application, a controller, operating memory, and a communications interface, wherein the server may be accessible via a network. The fertility/reproductive health system may further include a data store in communication with the server; and wherein the controller is configured to execute stored program instructions, that may include providing access to a user and one or more provider entities; receiving health data of a user; processing the received health data of the user; generating notifications to and/or from one or both of the user and the one or more provider entities; and generating fertility/reproductive treatment suggestions based on the processed health data of the user. The method may further include receiving and processing the health data of the user; instructing the user when to conduct certain lab tests; receiving and processing the lab test results of the user; analyzing the processed health data and the processed lab test results in comparison to a set of fertility/reproductive treatment protocols stored in the data store; and generating fertility/reproductive treatment suggestions based on the analyzed processed health data lab test results of the user in comparison to a set of fertility/reproductive treatment protocols stored in the data store. The method may further include presenting the generated fertility/reproductive treatment suggestions to a healthcare provider, and wherein the healthcare provider consults with the user on a recommended treatment plan. The health data of the user may include one or more of health data input directly by the user, responses to fertility/reproductive health intake protocols by the user, and/or health data of the user received from one or more provider entities. The lab test results may be received from one or more of the user, laboratory service provider, and/or healthcare provider. The healthcare provider may include a telehealth provider. The lab tests may be either or both of at-home or at-lab lab tests. The method may further include, prior to instructing the user when to conduct certain lab tests, sending the user a fertility health system kit. The fertility health system kit may include one or more blood spot cards; one or more ovulation tests; and one or more pregnancy tests. The one or more of the lab test results may include test results for one or more of anti-mullerian hormone (AMH), thyroid-stimulating hormone (TSH), follicle-stimulating hormone (FSH), estradiol (E2), luteinizing hormone (LH), progesterone (Pg), and total testosterone (TT), thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), and/or other hormones potentially related to fertility/reproductive health. The one or more of the lab tests may include tests for one or more of saliva testing for men or women and/or semen analysis including one or more of semen volume, concentration, count, motility, total motile count, morphology and/or DNA fragmentation.

In still yet another embodiment, a fertility/reproductive health system kit is provided. The fertility health system kit may include one or more blood spot cards; one or more ovulation tests; and/or one or more pregnancy tests.

### BRIEF DESCRIPTION OF DRAWINGS

Having thus described the subject matter of the invention in general terms, reference will now be made to the accompanying drawings, which are not necessarily drawn to scale, and wherein:

FIG. 1 illustrates a block diagram of an example of a fertility/reproductive health system, in accordance with an embodiment of the invention;

FIG. 2 illustrates a block diagram of an example of a fertility/reproductive health kit of the fertility/reproductive health system, in accordance with an embodiment of the invention;

FIG. 3 illustrates one example instantiation of the fertility/reproductive health kit shown in FIG. 2, in accordance with an embodiment of the invention;

FIG. 4A and FIG. 4B illustrate a flow diagram of an example of a method of using the fertility/reproductive health system, in accordance with an embodiment of the invention; and

FIG. 5 through FIG. 33 illustrates example screenshots of an example of a fertility/reproductive health system mobile app for use in the fertility/reproductive health system, in accordance with an embodiment of the invention.

### DETAILED DESCRIPTION

The subject matter of the invention now will be described more fully hereinafter with reference to the accompanying drawings, in which some, but not all embodiments of the subject matter of the invention are shown. Like numbers refer to like elements throughout. The subject matter of the invention may be embodied in many different forms and should not be construed as limited to the embodiments set forth herein; rather, these embodiments are provided so that this disclosure will satisfy applicable legal requirements. Indeed, many modifications and other embodiments of the subject matter of the invention set forth herein will come to mind to one skilled in the art to which the subject matter of the invention pertains having the benefit of the teachings presented in the foregoing descriptions and the associated drawings. Therefore, it is to be understood that the subject matter of the invention is not to be limited to the specific embodiments disclosed and that modifications and other embodiments are intended to be included within the scope of the appended claims.

In some embodiments, the subject matter of the invention provides a system, protocols, and methods for assisting fertility/reproductive health for men and/or women, including, but not limited to, fertility health, trimester-one pregnancy health, and/or postpartum health. For ease of reference the term fertility health is used herein below, but refers to fertility and/or reproductive health, including, but not limited to, fertility health, trimester-one pregnancy health, and/or postpartum health. While primarily described below for women, the system, protocols, and methods disclosed herein should be understood to apply to assisting fertility/reproductive health for men as well.

In some embodiments, the disclosed fertility health system, protocols, and methods provide a networked computing system that may include a fertility health application that may be based on certain clinical protocols, such as, but not limited to, certain fertility health intake protocols, certain fertility treatment protocols, and/or certain fertility SOAP (Subjective, Objective, Assessment, and Plan) notes, for guiding users and/or healthcare providers with respect to assisting the fertility and/or reproduction health of the users.

In some embodiments, the disclosed fertility health system, protocols, and methods provide a networked computing system that may accessed by multiple entities, such as, but not limited to, users seeking assistance with respect to fertility and/or reproduction health, telehealth providers, laboratory service providers, pharmacies, payment processing entities, IVF providers, and the like.

In some embodiments, the disclosed fertility health system, protocols, and methods provide a networked computing system that may be accessed by multiple entities using, for example, a fertility health system mobile app.

In some embodiments, the disclosed fertility health system, protocols, and methods provide a networked computing system including a fertility health application and a fertility health system mobile app that may be used throughout the fertility health intake protocols and fertility treatment protocols for ongoing communication between entities of fertility health system; for example, push notifications may be automatically generated to users and confirmation notifications may be returned from users to the fertility health application.

In some embodiments, the disclosed fertility health system, protocols, and methods provide a networked computing system including a fertility health application, a fertility health system mobile app, and may further include an at-home fertility test kit by which labs may be collected from the user and wherein the fertility test kit may include, for example, a blood spot card for dried blood spot (DBS) (i.e., finger prick) tests, multiple ovulation test strips, and/or multiple pregnancy test strips.

In some embodiments, the disclosed fertility health system, protocols, and methods may further include an at-home fertility test kit that may be used to test for multiple hormones that may include both hormones directly related to fertility and other hormones that conventional at-home fertility testing solutions commonly overlook. In one example, the selected hormones to be tested for may include, but are not limited to, anti-mullerian hormone (AMH), thyroid-stimulating hormone (TSH), follicle-stimulating hormone (FSH), estradiol (E2), luteinizing hormone (LH), progesterone (Pg), and total testosterone (TT). In addition, the selected hormones to be tested for may include thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), and/or other hormones potentially related to fertility/reproductive health. Lab tests may include tests for one or more of saliva testing for men or women and/or semen analysis including one or more of semen volume, concentration, count, motility, total motile count, morphology and/or DNA fragmentation.

In some embodiments, the disclosed fertility health system, protocols, and methods use (1) self-reported clinical information from users, (2) lab results, and (3) certain clinical protocols, such as, but not limited to, certain fertility health intake protocols, certain fertility treatment protocols, and/or certain fertility SOAP notes, to generate a treatment and prescribing plan and specific therapies for the patient or subject.

In some embodiments, the disclosed fertility health system, protocols, and methods may be used to scrub (1) patient self-reported information against (2) lab results and against (3) certain clinical protocols (e.g., certain fertility health intake protocols, certain fertility treatment protocols, and/or certain fertility SOAP notes) to create a customized treatment plan for the patient or subject. Further, machine learning may be used to optimize the diagnosis and therapies provided to the patient or subject.

Further, a method is provided of using the disclosed fertility health system, fertility health application, fertility health system mobile app, fertility health intake protocols, fertility treatment protocols, and/or fertility SOAP notes with respect to assisting the fertility and/or reproduction health of the users.

In some embodiments, the disclosed fertility health system, protocols, and methods provide a platform designed for identifying and treating fertility issues based on received assessment and heuristic data, which is employed to generate diagnoses based on protocols (e.g., fertility health intake protocols, fertility treatment protocols, and/or fertility SOAP notes), treatment plans, and specific therapies for the patient.

Additionally, the disclosed fertility health system, protocols, and methods for assisting fertility health as described herein is not limited to supporting fertility health only. The fertility health system, protocols, and methods for assisting fertility health may be suitable to aid other health issues, such as, but not limited to, trimester-one pregnancy issues and/or postpartum issues. For example, the clinical protocols, such as, but not limited to, certain fertility health intake protocols, certain fertility treatment protocols, and/or certain fertility SOAP notes, may be used for guiding users and/or healthcare providers with respect to assisting trimester-one pregnancy issues and/or postpartum issues.

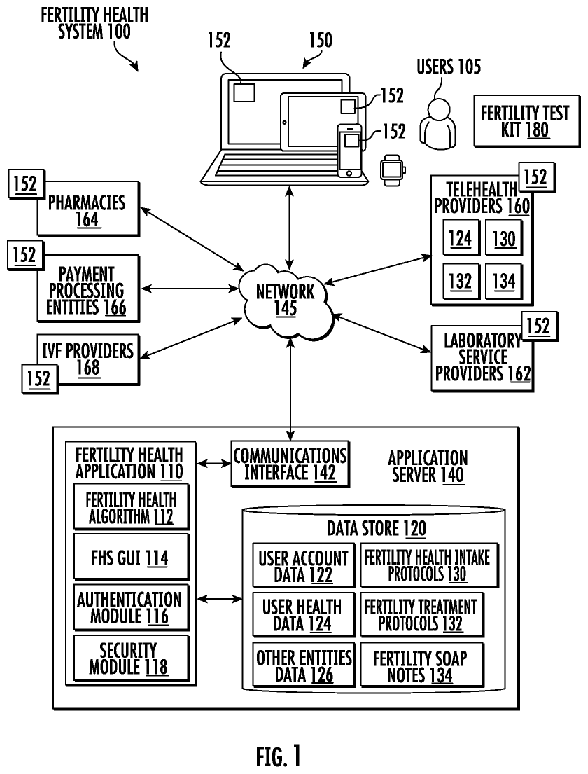

Referring now to FIG. 1 is a block diagram of an example of a fertility health system 100, in accordance with an embodiment of the invention. In this example, fertility health system 100 may be provided in a networked computing configuration that includes a fertility health application 110 and a data store 120 running on an application server 140.

At application server 140, fertility health application 110 may further include fertility health algorithm 112, a fertility health system (FHS) graphical user interface (GUI) 114, an authentication module 116, and a security module 118. Further, user account data 122, user health data 124, other entities data 126, fertility health intake protocols 130, fertility treatment protocols 132, and fertility SOAP notes 134 may be stored at data store 120. Additionally, application server 140 may be accessible via a network 145. Network 145 may be, for example, a local area network (LAN) and/or a wide area network (WAN) for connecting to the Internet or to an Intranet. Application server 140 may connect to network 145 by any wired and/or wireless means. Application server 140 may include, for example, a communications interface 142.

A plurality of users 105 may be associated with the fertility health system 100. In fertility health system 100, users 105 may be any women or men seeking help in the area of fertility and/or reproduction health. The users 105 may access fertility health application 110 at application server 140 via their respective user computers 150 and network 145. User computers 150 may be any computing device, such as, but not limited to, a desktop computer, a laptop computer, a handheld computing device, a mobile phone (or smart phone), a tablet device, a smartwatch, and the like. Any information about users 105 may be stored in user account data 122 and/or user health data 124 at data store 120 of application server 140. For example, a user profile for each user 105 in user account data 122 may include, for example, account information, user name, user group name, user/group credentials, user payment information, and the like.

In one example, users 105 may interact with the fertility health application 110 using FHS GUI 114 at application server 140. In this example, FHS GUI 114 may be a web-based GUI that is accessible via network 145. For example, fertility health application 110 at application server 140 may be a software application that may be implemented as a web application and run in a web browser, such as Google Chrome or Microsoft Edge.

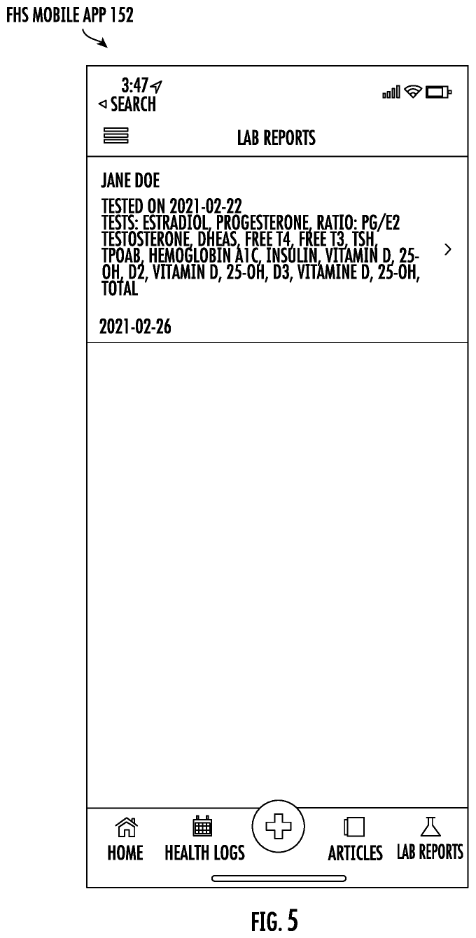

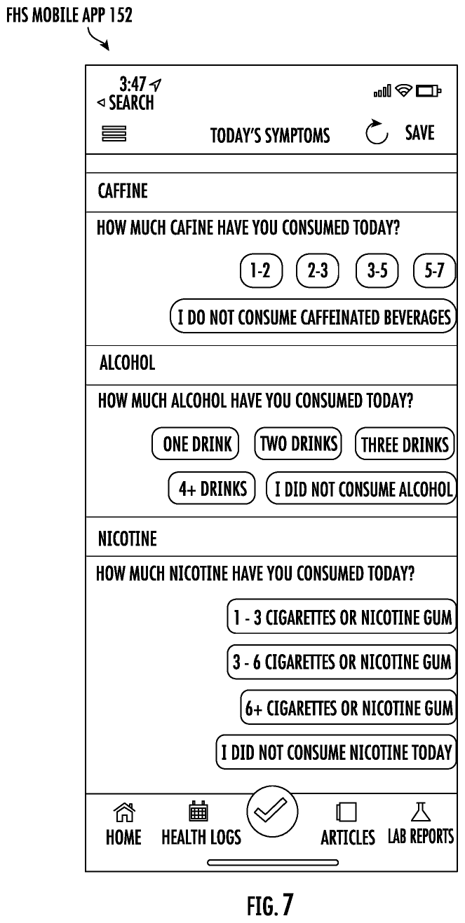

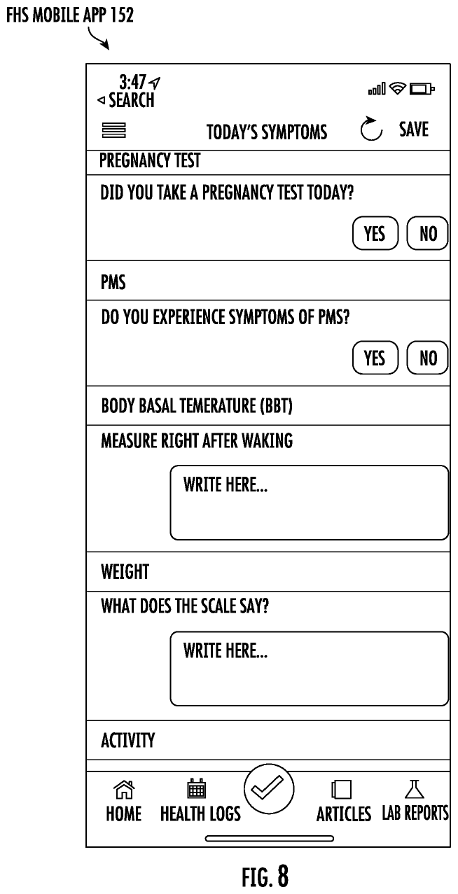

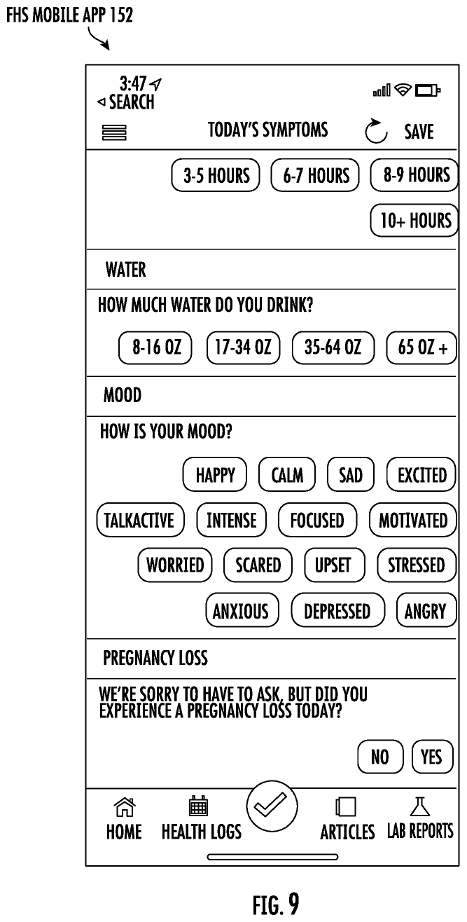

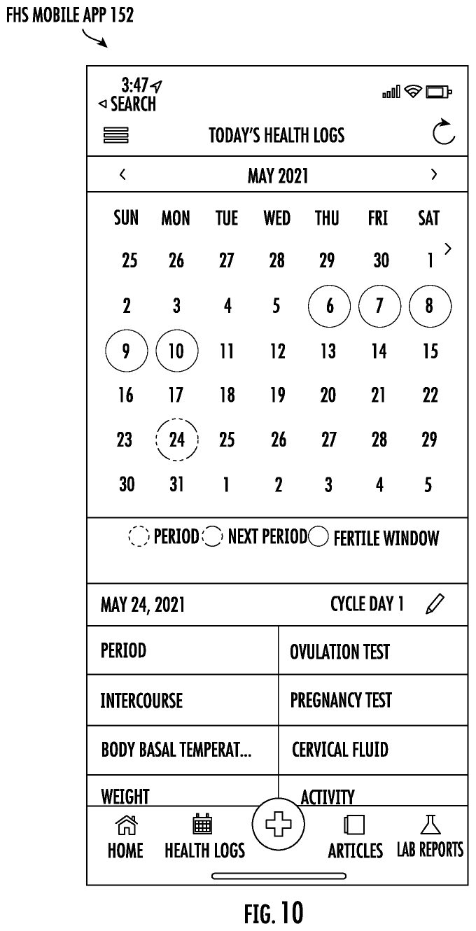

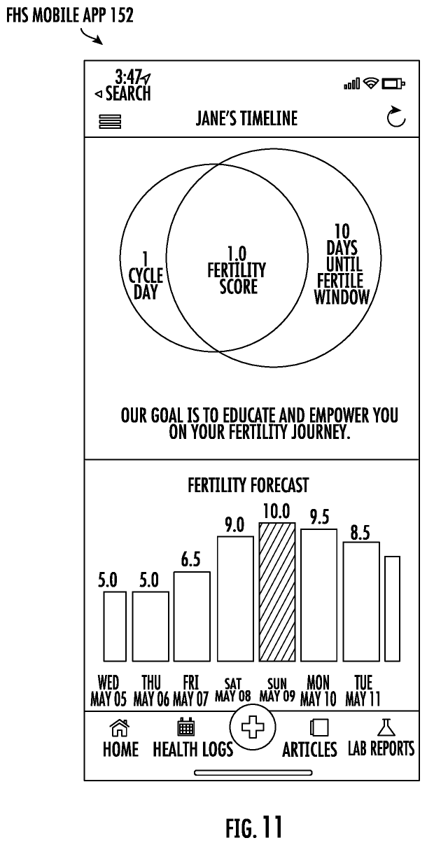

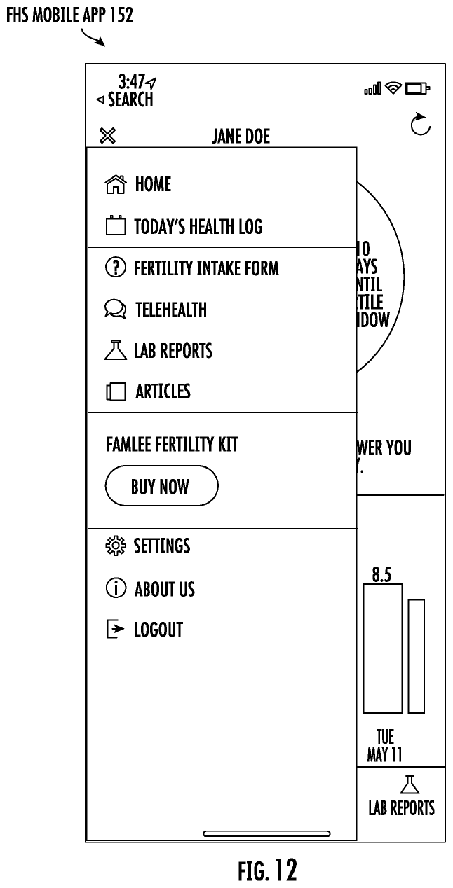

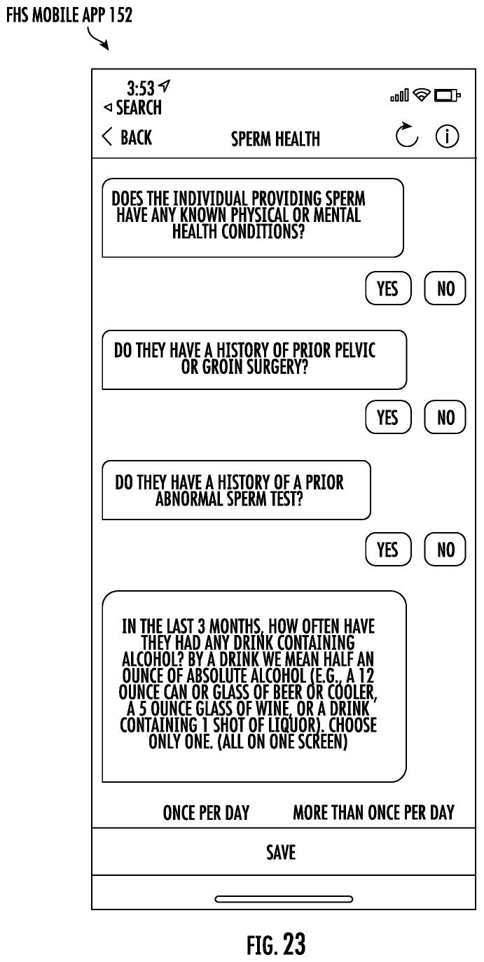

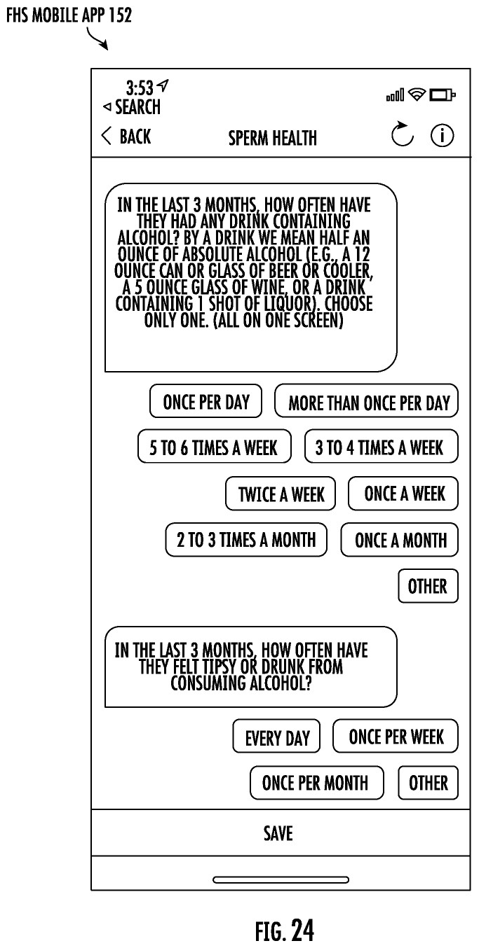

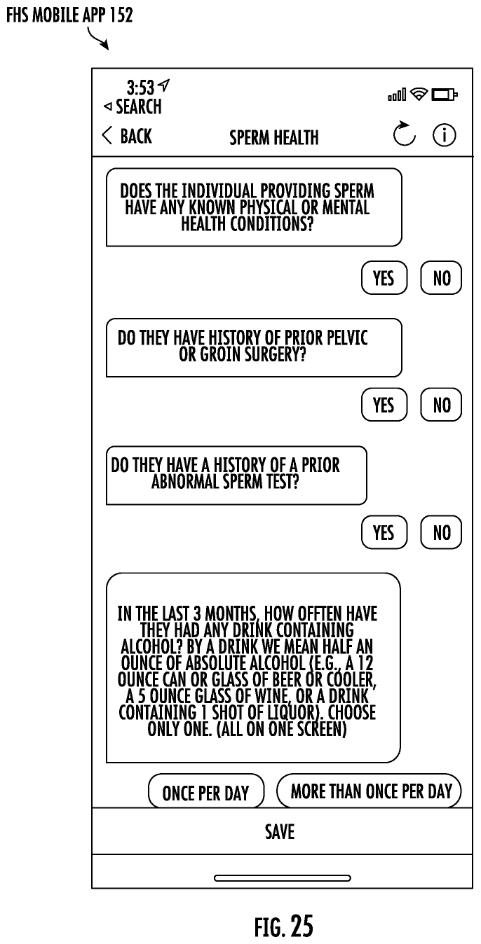

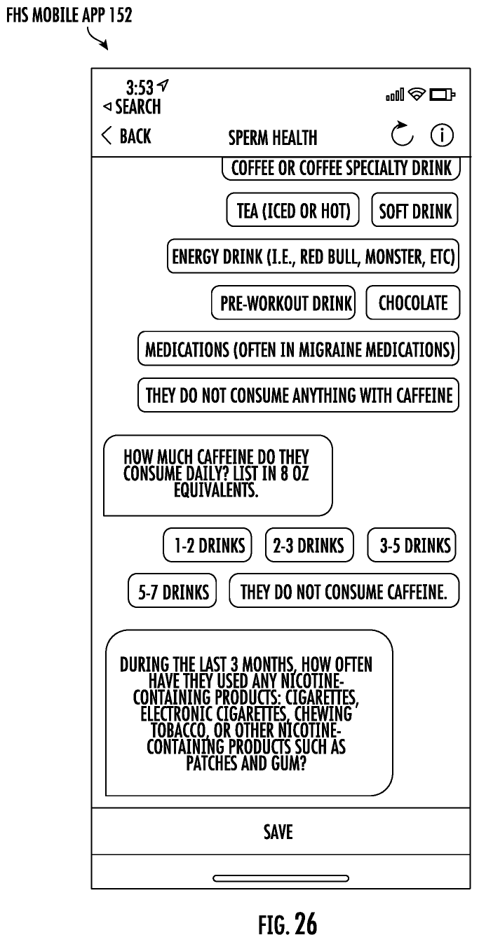

A FHS desktop application 152 or FHS mobile app 152 may be installed and running on each of the user computers 150. FHS desktop application 152 and FHS mobile app 152 is hereafter called FHS mobile app 152. In this example, FHS mobile app 152 may be implemented, for example, as a .NET application, a desktop application, a mobile app, an application program interface (API), and the like. When configured as a mobile app, FHS mobile app 152 may be designed to operate on any device platform, including for example, Windows, Android, Apple, and the like. Accordingly, users 105 may interact with the fertility health application 110 using FHS mobile app 152 of their user computer 150 (e.g., smart phone or tablet device). Example screenshots of FHS mobile app 152 are shown and described hereinbelow with reference to FIG. 5 through FIG. 33.

Referring still to FIG. 1, fertility health system 100 may also include one or more healthcare providers, such as telehealth providers 160, one or more laboratory service providers 162, one or more pharmacies 164, one or more payment processing entities 166, and one or more IVF providers 168. Further, the healthcare providers, e.g., telehealth providers 160, laboratory service providers 162, pharmacies 164, payment processing entities 166, IVF providers 168, and/or other authorized providers and entities may access fertility health application 110 via FHS mobile app 152.

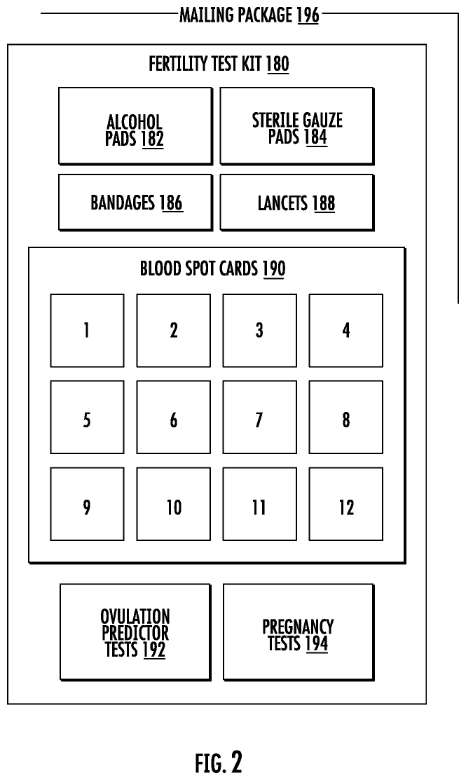

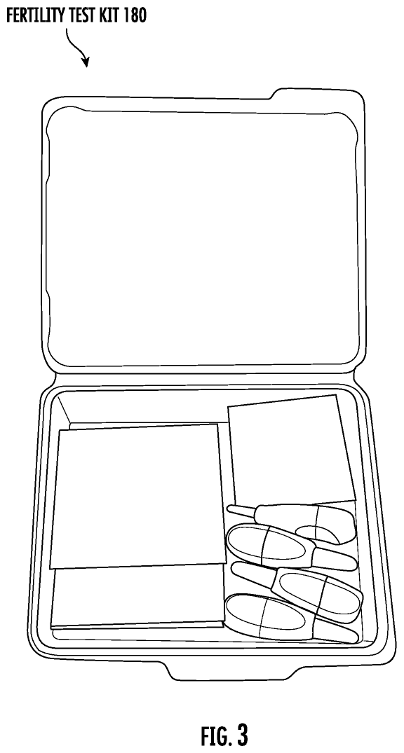

Additionally, in fertility health system 100, a fertility test kit 180 may be provided to any user 105. Fertility test kit 180 may include, for example, a blood spot card for dried blood spot (DBS) (i.e., finger prick) tests, multiple ovulation test strips, multiple pregnancy test strips, and/or other tests and related items. For example, FIG. 2 is a block diagram of an example of fertility test kit 180 of fertility health system 100. Further, FIG. 3 shows an example of one instantiation of an example fertility test kit 180 shown in FIG. 2. In this example, fertility test kit 180 may be an at-home test kit that may include multiple alcohol pads 182, multiple sterile gauze pads 184, multiple bandages 186, multiple lancets 188, multiple blood spot cards 190, multiple ovulation predictor tests 192 (e.g., urine test strips), and/or the multiple pregnancy tests 194 (e.g., urine test strips). The ovulation predictor tests 192 (also known as ovulation predictor kits (OPKs)) may use a small urine sample to detect a luteinizing hormone (LH) surge. The LH surge may be an indicator that user 105 may be approaching ovulation. Fertility test kit 180 may include in the alternative, or in addition at-lab test kit items for the user to use for testing at a laboratory service provider's 162 or healthcare provider's facility.

Each blood spot card 190 may be used to collect up to, in one non-limiting example, about twelve DBSs (i.e., via finger prick). As a non-limiting example, one blood spot card 190 may be used to test for one or more of anti-mullerian hormone (AMH), thyroid peroxidase antibody (TPoAB), free triiodothyronine (FT3), free thyroxine (FT4), fasting insulin (FI), hemoglobin A1C (HgbA1c), thyroid-stimulating hormone (TSH), dehydroepiandrosterone sulfate (DHEA-s), follicle-stimulating hormone (FSH), estradiol (E2), vitamin D (Vit D), and luteinizing hormone (LH) on a certain cycle day. Then, another blood spot card 190 may be used to test for progesterone (Pg), and total testosterone (TT) on a different cycle day.

Referring now again to FIG. 1, accordingly, to support fertility health intake protocols 130, fertility treatment protocols 132, and fertility SOAP notes 134, fertility test kit 180 may provide a mechanism by which a user 105 may be tested for multiple hormones that may include both hormones directly related to fertility and other hormones that conventional at-home fertility testing solutions commonly overlook. In fertility health system 100, in one example, the selected hormones to be tested for may include, but are not limited to, one or more of anti-mullerian hormone (AMH), thyroid-stimulating hormone (TSH), follicle-stimulating hormone (FSH), estradiol (E2), luteinizing hormone (LH), progesterone (Pg), and total testosterone (TT). In addition, the selected hormones to be tested for may include thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), and/or other hormones potentially related to fertility/reproductive health. The lab tests may include tests for one or more of saliva testing for men or women and/or semen analysis including one or more of semen volume, concentration, count, motility, total motile count, morphology and/or DNA fragmentation. The system and method may further include at-home phlebotomy, walk in labs, at-home or walk in semen analysis and saliva testing for men and women. Again, an example of fertility test kit 180 is shown and described hereinabove with respect to FIG. 2 and FIG. 3.

With respect to fertility health system 100, telehealth providers 160 may be any healthcare providers with expertise in fertility and/or reproduction health. Using FHS mobile app 152, telehealth providers 160 may have access to user health data 124, fertility health intake protocols 130, fertility treatment protocols 132, and fertility SOAP notes 134. Laboratory service providers 162 may be any entities providing lab services (e.g., lab tests performed on specimens) that may be used to support healthcare services. Pharmacies 164 may be any entities where medicinal drugs are dispensed and sold. Examples of pharmacies may include, but are not limited to, Walgreens (Deerfield, Ill.), CVS (Woonsocket, R.I.), Right Aid (Camp Hill, Pa.), mail-order pharmacies, or any other pharmacy. Payment processing entities 166 may be, for example, any companies that process debit and/or credit cards, and/or any other companies for exchanging funds, such as PayPal® (San Jose, Calif.) and Zelle®. IVF providers 168 may be any entities that provide IVF services.

Authentication module 116 of fertility health application 110 may be used to manage the authentication process of any entities of fertility health system 100, such as users 105, telehealth providers 160, laboratory service providers 162, pharmacies 164, payment processing entities 166, and IVF providers 168. For example, when the user 105 signs into the fertility health application 110, a standard authentication process may be performed that allows the user 105 to access fertility health application 110. User-sign in may occur a number of ways. In one example, the user 105 may use a web browser to access FHS GUI 114 of fertility health application 110 and enter credentials (e.g., username and password).

In another example, the user 105 may use FHS mobile app 152 of their user computer 150 to enter his/her credentials. In yet another example, the user sign-in process may occur automatically when the user 105 starts FHS mobile app 152. As users 105 are authorized to access fertility health system 100, user information may be stored in user account data 122 in data store 120. Similarly, as other entities (e.g., telehealth providers 160, laboratory service providers 162, pharmacies 164, payment processing entities 166, and IVF providers 168) access fertility health system 100, information may be stored in other entities data 126 in data store 120.

Security module 118 of fertility health application 110 may be used to perform any system security functions with respect to keeping secure the contents of data store 120 and/or any other information with respect to fertility health system 100. Security module 118 may use standard security techniques, such as encryption, secure hashtags (or hash tags), and the like. Data store 120 may be, for example, data repositories (like databases) and/or flat files that can store data. Further, fertility health system 100 is not limited to one data store 120 only. Fertility health system 100 may include multiple data stores 120. Further, data store 120 may be provided on a data server that is separate from application server 140.

Communications interface 142 at application server 140 may be any wired and/or wireless communication interface for connecting to a network (e.g., network 145) and by which information may be exchanged with other devices connected to the network. Examples of wired communication interfaces may include, but are not limited to, USB ports, RS232 connectors, RJ45 connectors, Ethernet, and any combinations thereof. Examples of wireless communication interfaces may include, but are not limited to, an Intranet connection, Internet, ISM, Bluetooth® technology, Bluetooth® Low Energy (BLE) technology, Wi-Fi, Wi-Max, IEEE 402.11 technology, ZigBee technology, Z-Wave technology, 6LoWPAN technology (i.e., IPv6 over Low Power Wireless Area Network (6LoWPAN)), ANT or ANT+ (Advanced Network Tools) technology, radio frequency (RF), Infrared Data Association (IrDA) compatible protocols, Local Area Networks (LAN), Wide Area Networks (WAN), Shared Wireless Access Protocol (SWAP), any combinations thereof, and other types of wireless networking protocols.

Referring still to FIG. 1, fertility health system 100 may operate in a client/server computing architecture, which is well known. In this example, fertility health application 110 at the application server 140 may be the server component of fertility health system 100, while FHS mobile app 152 at each of the user computers 150 may be the client component of fertility health system 100. In other words, FHS mobile app 152 at each of the user computers 150 is the counterpart to fertility health application 110 at application server 140.

Additionally, application server 140 may be any networked computing configuration as long as it is accessible via network 145 by other entities of fertility health system 100, such as users 105, telehealth providers 160, laboratory service providers 162, pharmacies 164, payment processing entities 166, and IVF providers 168. For example, fertility health system 100, and more particularly the fertility health application 110 on application server 140, may support a cloud computing environment. In a cloud computing environment, application server 140 may be the cloud server. Further, fertility health application 110 is not limited to running on one application server 140 only. Fertility health system 100 may include multiple application servers 140 (or cloud servers) in order to ensure high-availability of computing resources.

Generally, and referring still to FIG. 1, fertility health application 110 may be a software application that provides a means of using fertility health intake protocols 130, fertility treatment protocols 132, and FHS mobile app 152 to manage telehealth providers 160, laboratory service providers 162, pharmacies 164, payment processing entities 166, and IVF providers 168 to assist users 105 with respect to their fertility and/or reproduction health.

Additionally, using fertility health application 110 and FHS mobile app 152, ongoing communication between entities of fertility health system 100 may occur throughout fertility health intake protocols 130, fertility treatment protocols 132, and fertility SOAP notes 134. For example, using FHS mobile app 152, push notifications may be automatically generated to users 105 and confirmation notifications may be returned from users 105 to fertility health application 110 throughout the entirety of fertility health intake protocols 130, fertility treatment protocols 132, and fertility SOAP notes 134.

Fertility health algorithm 112 may be used to process all the information generated and/or received by fertility health application 110 and thereby manage the overall operations of fertility health system 100. In one example, fertility health algorithm 112 may be used to process user health data 124 with respect to fertility health intake protocols 130 and test results of fertility test kit 180 in order to determine the best intervention as indicated in fertility treatment protocols 132. Further, with respect to fertility health algorithm 112, machine learning may be used to optimize the diagnosis and therapies provided to the user 105 (or patient). For example, different types of assessment data may include, but is not limited to, layperson data, clinical data, biometric data, video data, medical data, heuristic date, and the like. Further, different types of assessment data may be weighted differently. Additionally, medical/healthcare providers may be granted access to the fertility health intake protocols 130, fertility treatment protocols 132, and/or fertility SOAP notes 134 of fertility health system 100 through the medical/healthcare provider's electronic medical records (EMR) system directly. Alternatively, the fertility health intake protocols 130, fertility treatment protocols 132, and/or fertility SOAP notes 134 may be integrated into a medical/healthcare provider's electronic medical records (EMR) system.

For example, fertility health intake protocols 130 of fertility health application 110 may include any user health information, which may be collected from a user 105 using FHS mobile app 152 and then stored in her/his user health data 124. For example, fertility health intake protocols 130 may include, but is not limited to, gynecological information, endocrine information, fertility information, intercourse or inseminations information, sperm health information, pregnancy history information, whole body health information, family history information, lifestyle information, and the like. Generally, the information collected in fertility health intake protocols 130 is provided to telehealth providers 160 only and not to users 105. Below is an example of a “USER INTAKE FORM” based on fertility health intake protocols 130. Using FHS mobile app 152, the USER INTAKE FORM may be filled out by a user 105. For example, the USER INTAKE FORM may include a series of questions in each topic area, as shown below. The below questions are examples only, and the USER INTAKE FORM should not be limited to the example questions listed below, and may include fewer, additional, and/or different questions.

Fertility treatment protocols 132 of fertility health application 110 may include treatment suggestions that telehealth providers 160 may make to users 105 based on symptoms and lab results and information in user health data 124 collected via the HEALTH INTAKE FORM or by any other means. Again, to support fertility health intake protocols 130, fertility treatment protocols 132, and/or fertility SOAP notes 134, the user 105 has been tested with respect to, for example, anti-mullerian hormone (AMH), thyroid peroxidase antibody (TPoAB), free triiodothyronine (FT3), free thyroxine (FT4), fasting insulin (FI), hemoglobin A1C (HgbA1c), thyroid-stimulating hormone (TSH), dehydroepiandrosterone sulfate (DHEA-s), follicle-stimulating hormone (FSH), estradiol (E2), vitamin D (Vit D), luteinizing hormone (LH), progesterone (Pg), and total testosterone (TT). Accordingly, based on this set of tests, an example of TREATMENT SUGGESTIONS of fertility health application 110 is shown below. The TREATMENT SUGGESTIONS are examples only, and should not be limited to the examples listed below, and may include fewer, additional, and/or different suggestions.

In fertility health system 100, fertility SOAP notes 134 of fertility health application 110 is provided to support and/or as a part of the clinical fertility health intake protocols 130 and/or fertility treatment protocols 132 that are used by telehealth providers 160 for treating users 105. Generally, SOAP notes are a standard method of documentation used by healthcare providers to write out notes in patient's charts and medical records. The SOAP note is the general cognitive framework for the medical team to use to assess patients.

For example, fertility SOAP notes 134 may provide the logic that uses the self-reported information from the USER INTAKE FORM of fertility health intake protocols 130. Then, fertility SOAP notes 134 scrubs the self-reported information against the clinical fertility health intake protocols 130 and fertility treatment protocols 132, to inform the telehealth provider 160 how to clinically serve the user 105 based off of the combination of (1) the user's self-reported intake answers, (2) the clinical fertility health intake protocols 130 and fertility treatment protocols 132, and (3) the lab results.

The fertility SOAP notes 134 may provide the structure that each telehealth provider 160 may use to serve each user 105 in an efficient manner In fertility health system 100, an example of fertility SOAP notes 134 is shown below. The below SOAP notes 134 are examples only, and should not be limited to the examples listed below, and may include fewer, additional, and/or different notes.

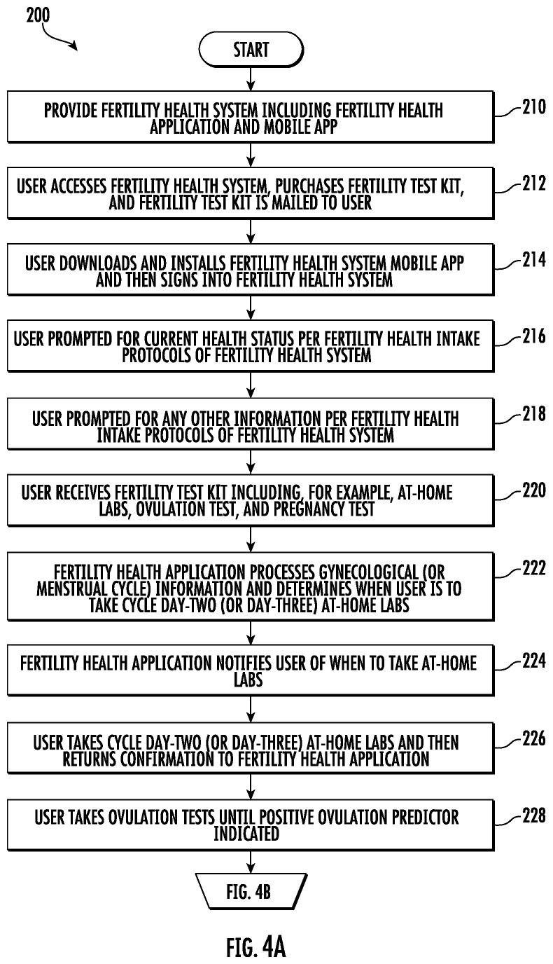

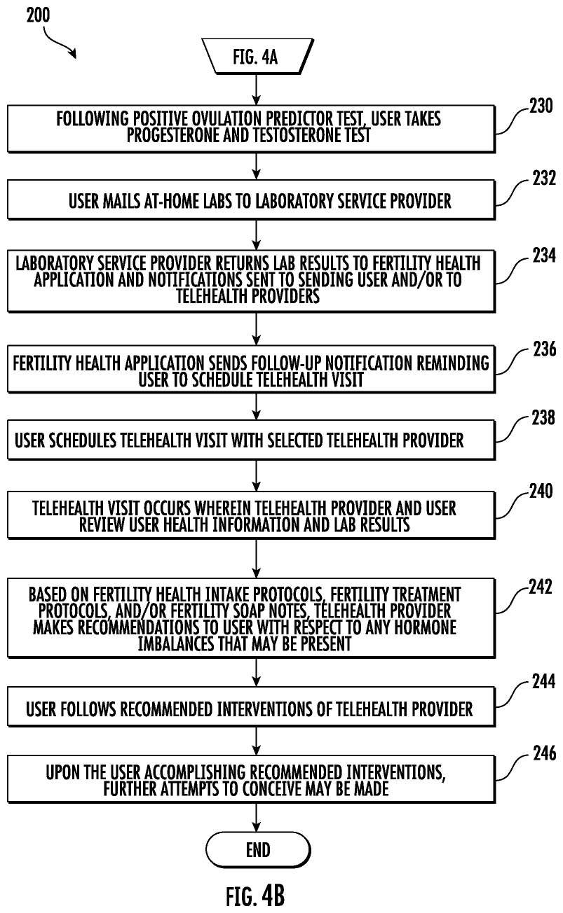

Referring now to FIG. 4A and FIG. 4B is a flow diagram of an example of a method 200 of using the fertility health system 100 for assisting fertility and/or reproduction health of users 105. Further, FIG. 5 through FIG. 33 show screenshots of an example of FHS mobile app 152 and demonstrating certain steps of method 200. Further, throughout the steps of method 200, ongoing communication may occur between entities of fertility health system 100. For example, throughout the steps of method 200 and although not specifically stated, using FHS mobile app 152, push notifications may be automatically generated to users 105 and confirmation notifications may be returned from users 105 to fertility health application 110. Method 200 may include, but is not limited to, the following steps, and may carried out in a different order than listed below.

At a step 210, the fertility health system including a fertility health application and a mobile app may be provided. For example, the fertility health system 100 is provided as described hereinabove with reference to FIG. 1 through FIG. 3. For example, fertility health system 100 may be provided and may include fertility health application 110 and FHS mobile app 152 that may use fertility health intake protocols 130, fertility treatment protocols 132, and/or fertility SOAP notes 134 to assist users 105 with respect to their fertility and/or reproduction health.

At a step 212, a user accesses the fertility health system, may purchase a fertility test kit, and the fertility test kit mailed to the user. For example, a certain user 105 may access the fertility health system 100 shown in FIG. 1. Then, the user 105 may purchase a fertility test kit 180, for example, as shown in FIG. 2 and FIG. 3. The purchase may be managed by a certain payment processing entity 166 of fertility health system 100. Then, the fertility test kit 180 may be mailed to the user 105. In an alternative embodiment, the fertility test kit may be ordered and/or sent/kitted from a third-party, such as a lab provider. In such an embodiment.

At a step 214, the user may download and install the fertility health system mobile app and then signs into the fertility health system. For example, the user 105 downloads and installs FHS mobile app 152 on her/his user computer 150 (e.g., smart phone, tablet device). Then, the user 105 may sign into fertility health system 100 and wherein the sign-in process may be managed by authentication module 116. Example screenshots of an example of FHS mobile app 152 are shown in FIG. 5 through FIG. 33.

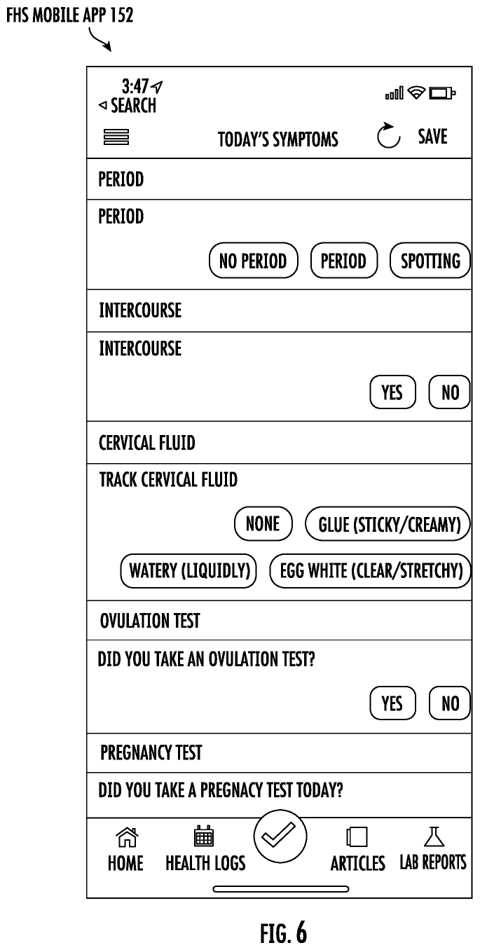

At a step 216, the user may be prompted for current health status per the fertility health intake protocols of the fertility health system. For example, using FHS mobile app 152, the user 105 may be prompted for “today's” health status wherein the user 105 may log any symptoms that they may be currently experiencing, as shown, for example, in FIG. 6 through FIG. 10.

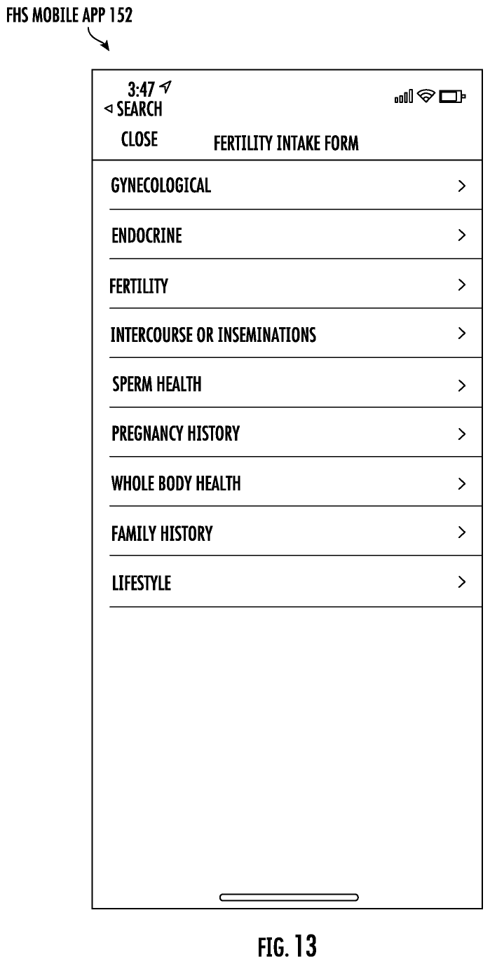

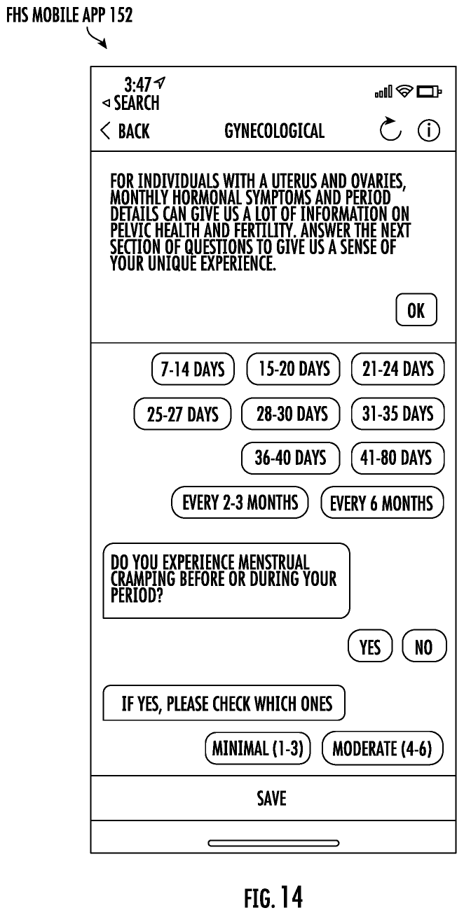

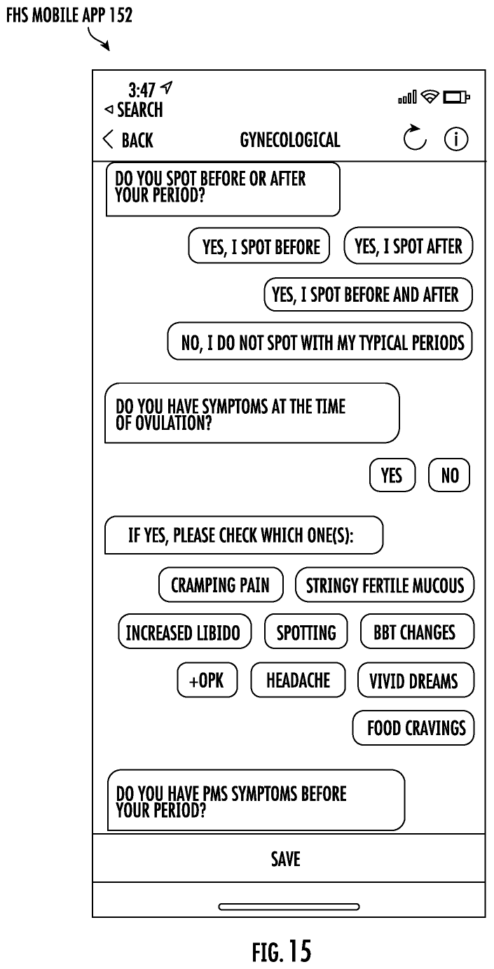

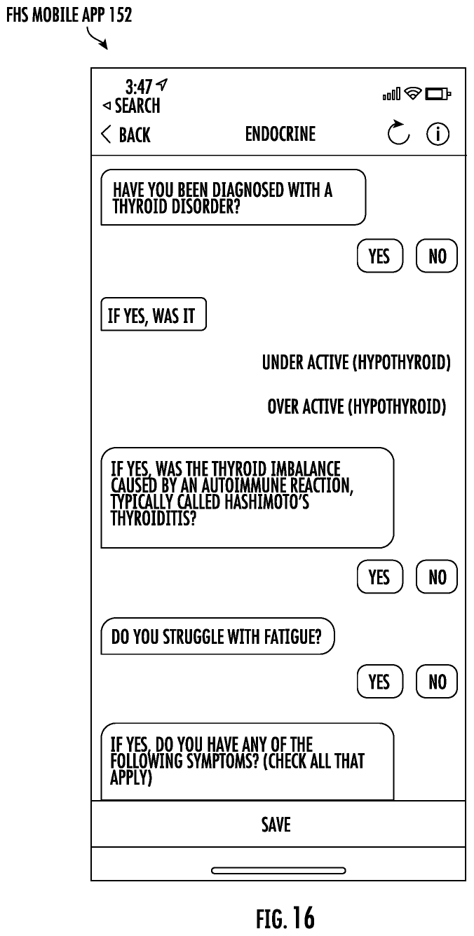

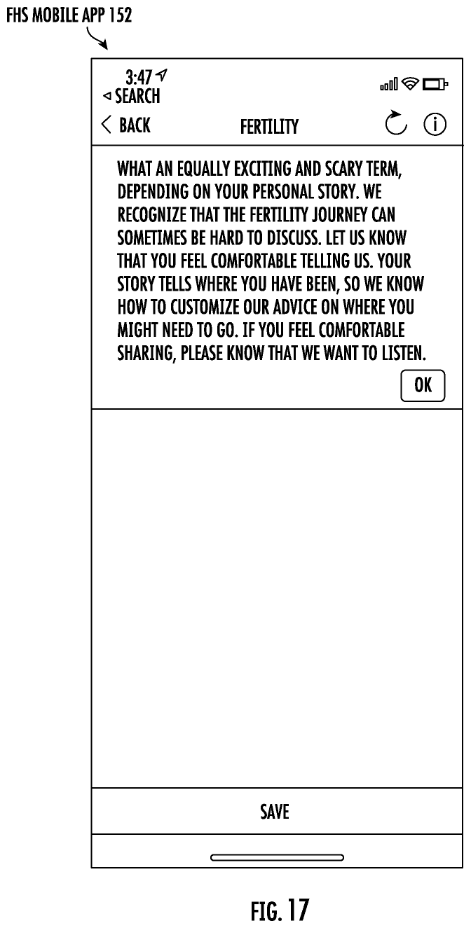

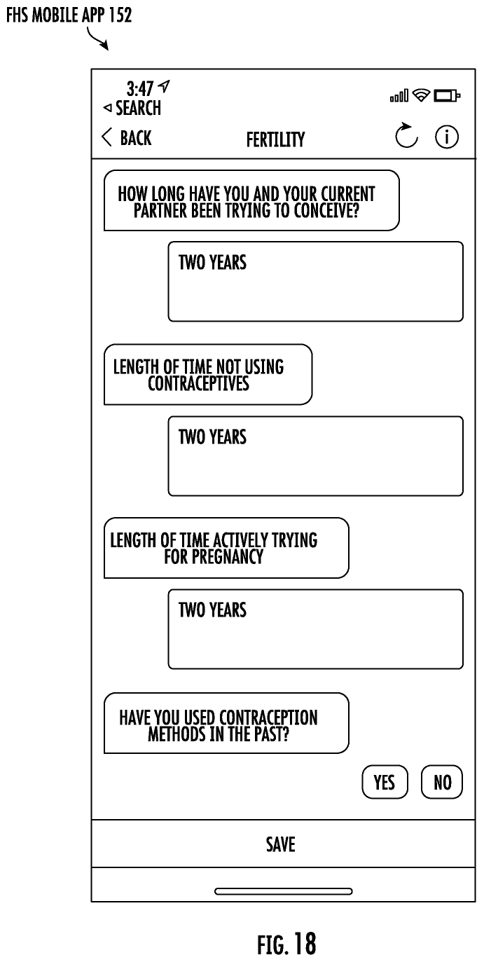

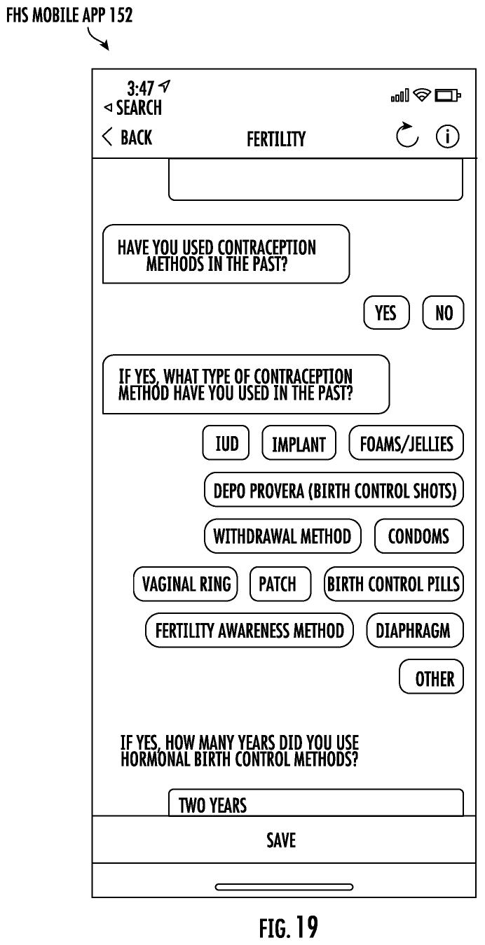

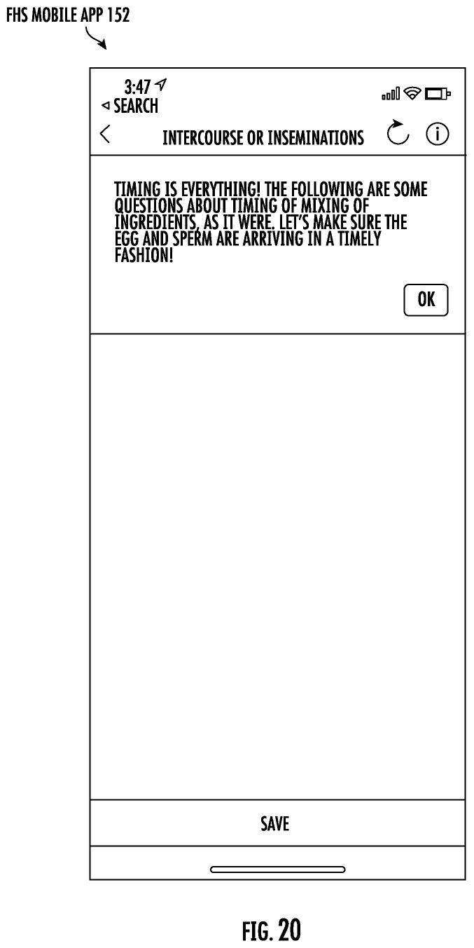

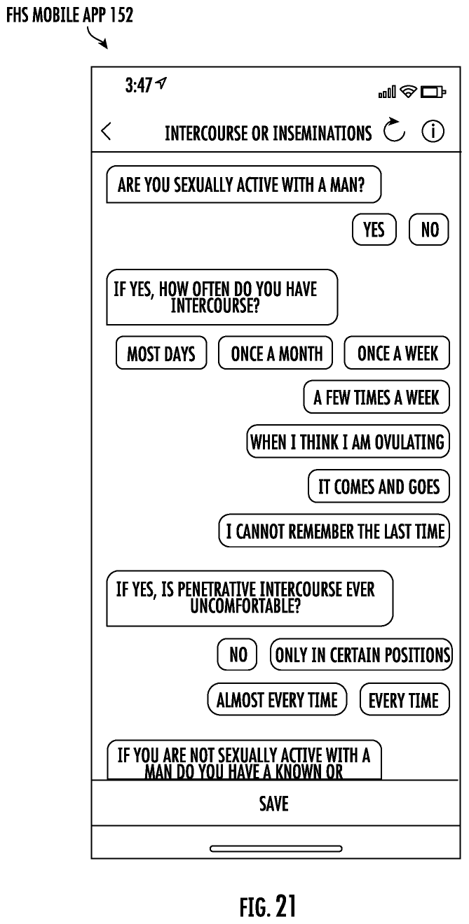

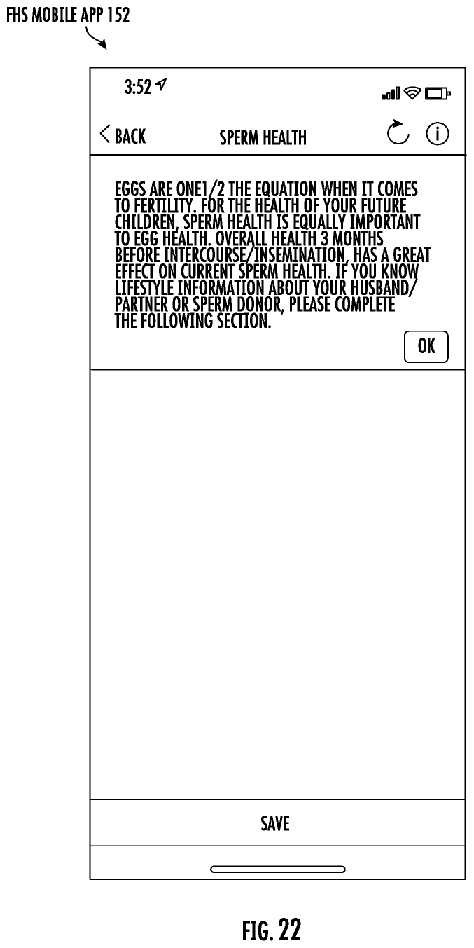

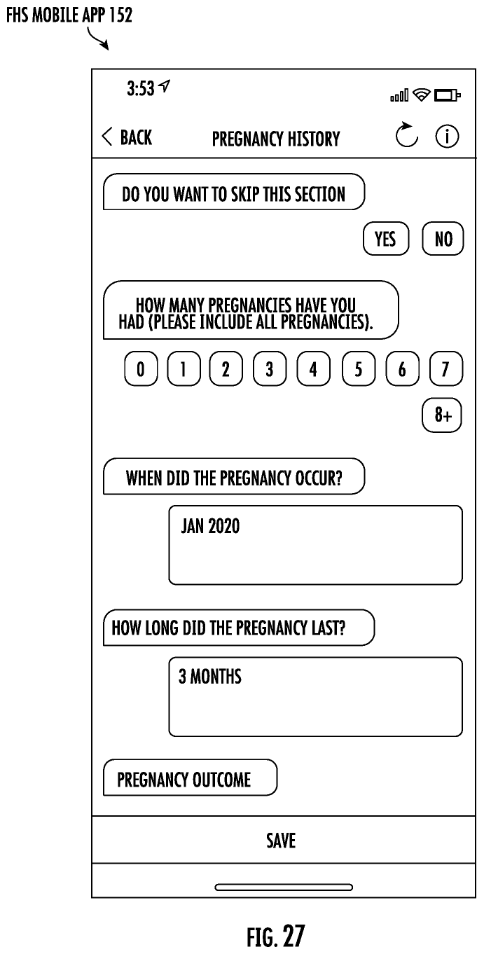

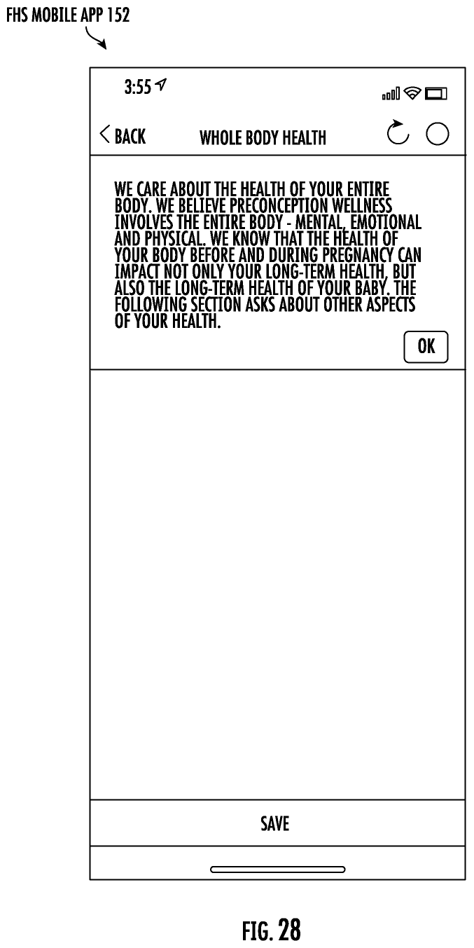

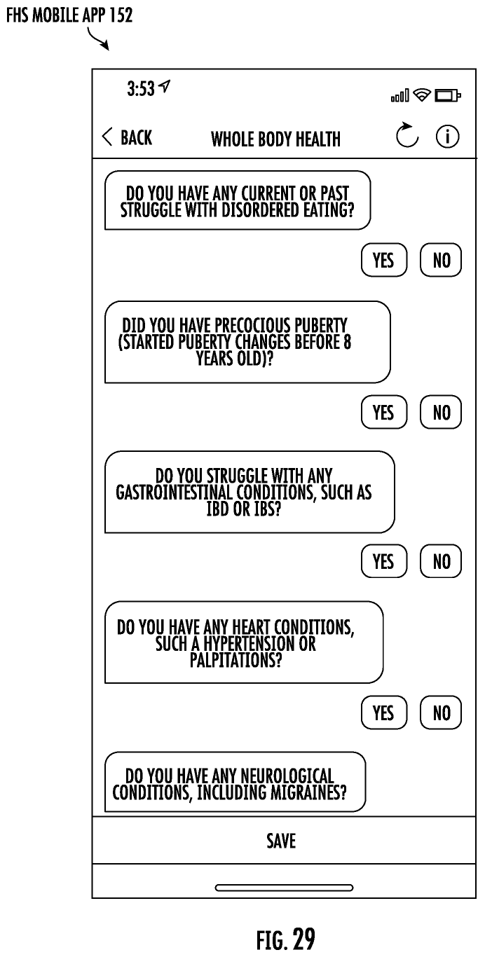

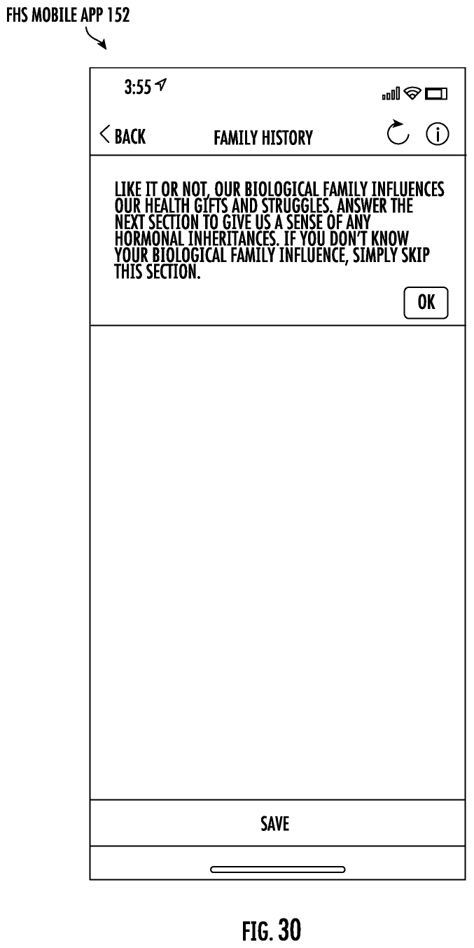

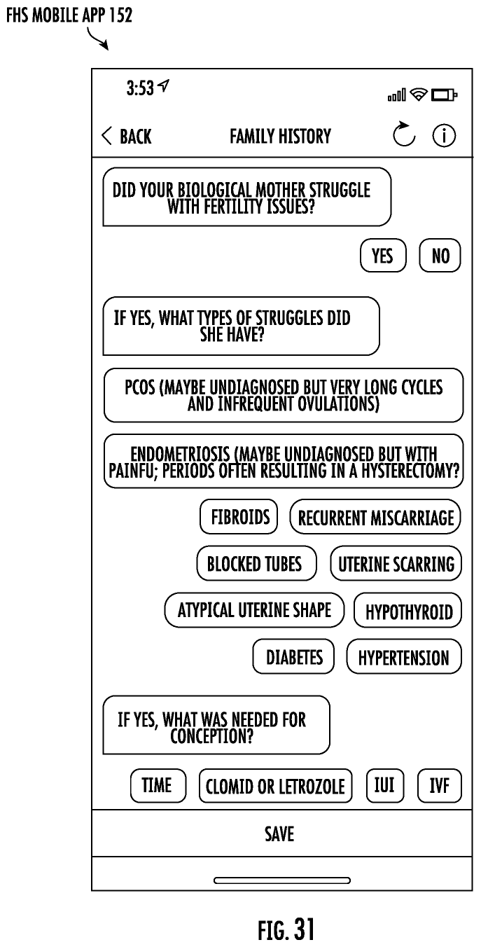

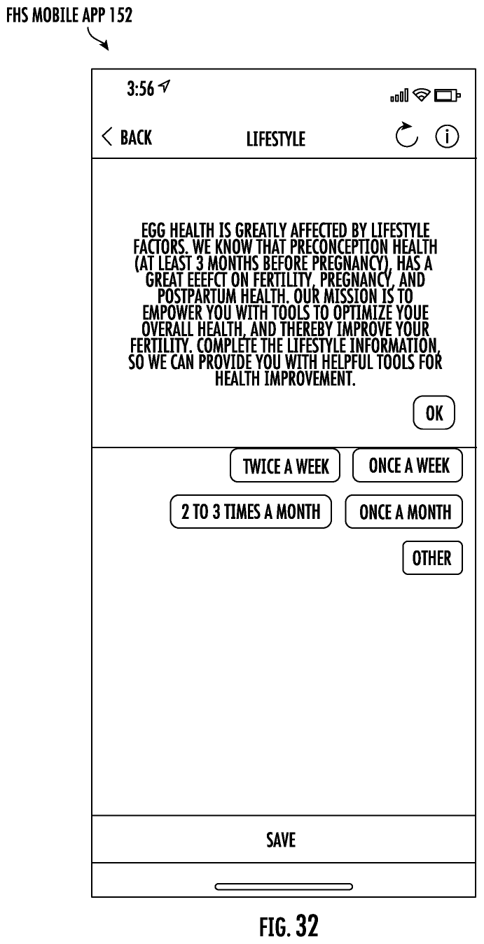

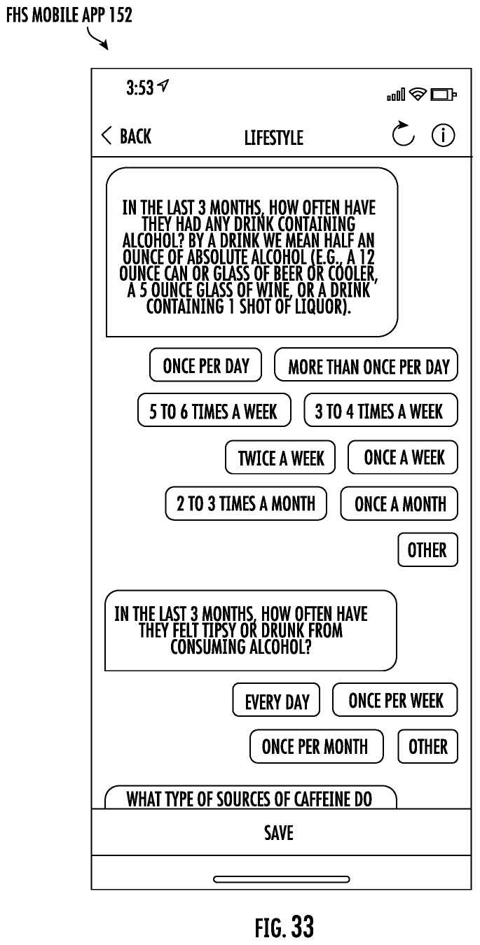

At a step 218, the user may be prompted for any other information per the fertility health intake protocols of the fertility health system. For example, using FHS mobile app 152 and the USER INTAKE FORM as described hereinabove, the user 105 may be prompted for any other information per fertility health intake protocols 130 of fertility health system 100. For example, the user 105 may be prompted for gynecological information, as shown, for example, in FIG. 13, FIG. 14, and FIG. 15 (also see the gynecological portion of USER INTAKE FORM and SOAP NOTES). Next, the user 105 may be prompted for endocrine information, as shown, for example, in FIG. 16 (also see the endocrine portion of USER INTAKE FORM and SOAP NOTES). Next, the user 105 may be prompted for fertility information, as shown, for example, in FIG. 17, FIG. 18, and FIG. 19 (also see the fertility portion of USER INTAKE FORM and SOAP NOTES). Next, the user 105 may be prompted for intercourse or inseminations information, as shown, for example, in FIG. 20 and FIG. 21 (also see the intercourse or inseminations portion of USER INTAKE FORM and SOAP NOTES). Next, the user 105 may be prompted for sperm health information, as shown, for example, in FIG. 22 through FIG. 26 (also see the sperm health portion of USER INTAKE FORM and SOAP NOTES). Next, the user 105 may be prompted for pregnancy history information, as shown, for example, in FIG. 27 (also see the pregnancy history portion of USER INTAKE FORM and SOAP NOTES). Next, the user 105 may be prompted for whole body health information, as shown, for example, in FIG. 28 and FIG. 29 (also see the whole body health portion of USER INTAKE FORM and SOAP NOTES). Next, the user 105 may be prompted for family history information, as shown, for example, in FIG. 30 and FIG. 31 (also see the family history portion of USER INTAKE FORM and SOAP NOTES). Next, the user 105 may be prompted for lifestyle information, as shown, for example, in FIG. 32 and FIG. 33 (also see the lifestyle portion of USER INTAKE FORM and SOAP NOTES).

The user data input into the fertility health system may include data from other sources, such as, but not limited to, fertility wearable devices (such as those offered by Mira, OuvSense, and/or the like).

At a step 220, the user may receive the fertility test kit including, for example, but not limited to, at-home labs, ovulation test, and pregnancy test. For example, the user 105 may receive fertility test kit 180, which may include the at-home labs, such as, but not limited to, blood spot cards 190 that may be used to collect multiple DBSs (i.e., via finger prick). Fertility test kit 180 may also include ovulation predictor tests 192 (e.g., urine test strips) and pregnancy tests 194 (e.g., urine test strips).

At a step 222, the fertility health application processes the user's data, for example, the fertility health application may process the user's gynecological (or menstrual cycle) information and then determines when the user is to take the labs, e.g., at-home labs. The labs may be at-home dried blood spot, at-home phlebotomy, and/or walk in clinic labs, and may also include at-home and at-clinic semen analysis and saliva testing for men and women. For example, fertility health application 110 processes the user 105's gynecological (or menstrual cycle) information and then determines when the user 105 is to take the at-home labs. For example, fertility health application 110 may determine what calendar day is day-two and day-three of the user 105's menstrual cycle.

At a step 224, the fertility health application notifies the user of when to take the cycle day-two (or day-three) at-home labs. For example, using FHS mobile app 152, on day-one of the user 105's menstrual cycle, fertility health application 110 notifies the user 105 to take the at-home labs on the morning of the next day, which is day-two. Note—if the user fails to take the at-home labs on day-two, fertility health application 110 may send another notification to the user 105 on day-two to take the at-home labs on the morning of the next day, which is now day-three.

At a step 226, the user takes the cycle day-two (or day-three) at-home labs and then returns confirmation to the fertility health application. For example, the user 105 takes the at-home labs of fertility test kit 180. That is, using the lancets 188, the user performs finger pricks to apply, for example, twelve drops of blood on the blood spot card 190, resulting in twelve DBSs. More specifically, user 105 takes at-home tests that correspond to lab tests for twelve of the fourteen hormones tested using fertility health system 100. In this step, the twelve hormones tested in the at-home labs may be, for example, anti-mullerian hormone (AMH), thyroid peroxidase antibody (TPoAB), free triiodothyronine (FT3), free thyroxine (FT4), fasting insulin (FI), hemoglobin A1C (HgbA1c), thyroid-stimulating hormone (TSH), dehydroepiandrosterone sulfate (DHEA-s), follicle-stimulating hormone (FSH), estradiol (E2), vitamin D (Vit D), luteinizing hormone (LH), and/or other hormones potentially related to fertility/reproductive health.

Then, upon completing the cycle day-two (or day-three) at-home labs, the user 105 uses FHS mobile app 152 to return a confirmation notification to fertility health application 110 that the cycle day-two (or day-three) at-home labs have been taken.

At a step 228, the user takes the ovulation test until a positive ovulation predictor is indicated. For example, at about cycle day-ten, the user 105 may start taking the ovulation predictor tests 192 (e.g., urine strip LH tests), wherein an LH surge may be an indicator that user 105 may be approaching ovulation. The ovulation predictor information is useful for guiding the user 105 with respect to taking the final two labs (e.g., Pg and IT labs).

At a step 230, in one example, seven (7) days following a positive ovulation predictor test, the user takes the progesterone test and the testosterone test, which are DBS tests and/or may be walk-in labs, or at-home phlebotomy. For example, seven (7) days following following a positive ovulation predictor test 192 at step 228, the user 105 uses a lancet 188 (i.e., finger pricks) and another blood spot card 190 and takes the progesterone (Pg) test and the testosterone (TT) test. In one example, for women who have standard 28-day menstrual cycles, then the user 105 may take the progesterone (Pg) and total testosterone (TT) test on cycle day-21. In another example, for women who don't have standard 28-day menstrual cycles, the user 105 may take the Pg test and TT test 7 days after a confirmed ovulation predictor test. In yet another example, for women who don't have standard 28-day menstrual cycles, the user 105 may take the Pg test and TT test 7 days before an anticipated period start day. For example, this scenario may be for those women who did not receive a positive ovulation predictor test. In this can, a woman may have to guess, using their knowledge of their menstrual cycle, 7 days before their estimated next menstrual cycle.

At a step 232, the user mails the at-home labs to the laboratory service provider. For example, the user 105 packages her blood spot card 190 into a mailing package 196 (see FIG. 2) provided with fertility test kit 180 and then mails the blood spot card 190 to one of the laboratory service providers 162 of fertility health system 100. The at-home labs may also include at-home phlebotomy and/or walk in clinic labs. In some instances, the user may not need to mail in the at-home labs, for example, if the lab test is conducted at the laboratory service provider's clinic. Further still, the lab tests may include tests for one or more of saliva testing for men or women and/or semen analysis including one or more of semen volume, concentration, count, motility, total motile count, morphology and/or DNA fragmentation.

At a step 234, the laboratory service provider returns the lab results to the fertility health application and notification thereof is sent to the user and/or the telehealth providers. For example, using FHS mobile app 152, a certain laboratory service provider 162 returns the lab results to fertility health application 110 and notification thereof is sent to the sending user 105 and/or to one or more telehealth providers 160. FIG. 5 shows an example of using FHS mobile app 152 to access and view a lab report.

At a step 236, the fertility health application sends a follow-up notification reminding the user to schedule a telehealth visit. For example, using FHS mobile app 152, fertility health application 110 sends a follow-up notification to remind the user 105 to schedule a telehealth visit with one of the telehealth providers 160 of fertility health system 100.

At a step 238, the user schedules a telehealth visit with a selected telehealth provider. For example, using FHS mobile app 152, the user 105 schedules a telehealth visit with one of the telehealth providers 160 of fertility health system 100.

At a step 240, a telehealth visit occurs wherein the telehealth provider and the user review the user health information and lab results that are based on the fertility health intake protocols 130. For example, using FHS mobile app 152, a telehealth visit occurs between the user 105 and the selected telehealth provider 160. Here, and using FHS mobile app 152, telehealth provider 160 may access and view the user 105's user health data 124, which includes information from the USER INTAKE FORM as well as all test results from the at-home labs of fertility test kit 180, wherein the health data and lab tests are based on fertility health intake protocols 130. Then, the telehealth provider 160 and the user 105 may review all of the user health information and lab results.

At a step 242, using fertility health intake protocols, fertility treatment protocols, and/or fertility SOAP notes of the fertility health system, the telehealth provider makes recommendations to the user with respect to any hormone imbalances that may be present. For example, based on the fertility health intake protocols 130, fertility treatment protocols 132, and/or fertility SOAP notes 134 of fertility health system 100, the telehealth provider 160 may make certain recommendations to the user 105 with respect to any hormone imbalances that may be present, and may also make prescription medication suggestions regarding how to improve ovulation.

In particular, in this step, fertility SOAP notes 134 (or SOAP NOTES above) may provide the logic that uses the self-reported information from the USER INTAKE FORM of fertility health intake protocols 130. Then, fertility SOAP notes 134 scrubs the self-reported information against the clinical fertility health intake protocols 130 and fertility treatment protocols 132, to inform the telehealth provider 160 how to clinically serve the user 105 based off of the combination of (1) the user's self-reported intake answers, (2) the clinical fertility health intake protocols 130 and fertility treatment protocols 132, and (3) the lab results.

For example, the “P” portion of fertility SOAP notes 134 (or SOAP NOTES above) focuses on treatment suggestions (protocols). More specifically, the “P” portion of fertility SOAP notes 134 may include, but is not limited to, supplement recommendations, Rx recommendations, additional lab order suggestions, information on how to decide when to refer out, sperm health protocols, dosing of Rxs to prescribe, dosing of supplements to prescribe, and so on. In one example, the telehealth provider 160 may make prescribe one or more certain medications to correct one or more certain hormone imbalances. The one or more prescriptions may be filled by one of the pharmacies 164 of fertility health system 100 and then mailed to the user 105. By way of example, to support ovulation, supplements to support ovulation could be suggested (e.g., melatonin, maca, myo-innostitol).

At a step 244, the user follows the recommended interventions of the telehealth provider. For example, the user 105 follows the recommended interventions of the telehealth provider 160, which may be, for example, taking a certain medication for a period of time.

At a step 246, upon the user accomplishing the recommended interventions, further attempts to conceive may be made. For example, upon the user 105 accomplishing the recommended interventions, such as correcting any hormone imbalances with medication, further attempts to conceive may be made.

Whereas the fertility health system 100, fertility health application 110, and/or method 200 may be useful for aiding a certain percent of users 105 experiencing fertility and/or reproduction health issues to conceive, the remaining percent of users 105 that may still be unable to conceive may choose to reach out to IVF providers 168. Accordingly, fertility health system 100 and method 200 including fertility test kit 180 may be positioned as step-therapy to IVF. The fertility health system 100 and its methods, may provide triage (e.g., initial lab work up and/or treatment) to IVF providers (e.g., reproductive endocrinologists) and to fertility networks.

In summary and referring now again to FIG. 1 through FIG. 33, the fertility health system 100, fertility health application 110, fertility health intake protocols 130, fertility treatment protocols 132, fertility SOAP notes 134, and method 200 may be used for guiding users 105 and/or healthcare providers, such as telehealth providers 160, with respect to assisting the fertility and/or reproduction health of the users.

Further, using FHS mobile app 152, fertility health system 100 may be accessed by multiple entities, such as, but not limited to, users 105 seeking assistance with respect to fertility and/or reproduction health, telehealth providers 160, laboratory service providers 162, pharmacies 164, payment processing entities 166, IVF providers 168, and the like.

Further, throughout the operations of fertility health system 100, fertility health application 110, and/or method 200, including fertility health intake protocols 130, fertility treatment protocols 132, and/or fertility SOAP notes 134, ongoing communication may occur between entities of fertility health system 100. For example, using fertility health application 110 and/or FHS mobile app 152, push notifications may be automatically generated to users 105 and confirmation notifications may be returned from users 105 to fertility health application 110.

Further, fertility health system 100, fertility health application 110, and/or method 200 provide the at-home fertility test kit 180 by which labs may be collected from the user 105 and wherein fertility test kit 180 may include, for example, a blood spot card for dried blood spot (DBS) (i.e., finger prick) tests and other test strips, and may include at-home phlebotomy, walk-in laboratory labs, at-home and at-clinic semen analysis and saliva testing for men and women.

Further, in fertility health system 100, fertility health application 110, and/or method 200, fertility test kit 180 may be used to test for multiple hormones that may include both hormones directly related to fertility and other hormones that conventional at-home fertility testing solutions commonly overlook. In one example, the selected hormones to be tested for may include, but are not limited to, anti-mullerian hormone (AMH), thyroid-stimulating hormone (TSH), follicle-stimulating hormone (FSH), estradiol (E2), luteinizing hormone (LH), progesterone (Pg), and total testosterone (TT). In addition, the selected hormones to be tested for may include thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), and/or other hormones potentially related to fertility/reproductive health.

Further, fertility health system 100, fertility health application 110, and/or method 200 provide a platform designed for identifying and treating fertility issues based on received assessment and heuristic data, which is employed to generate diagnoses based on protocols (e.g., fertility health intake protocols 130, fertility treatment protocols 132, fertility SOAP notes 134), treatment plans, and specific therapies for the user 105.

Further, fertility health system 100, fertility health application 110, and/or method 200 use (1) self-reported clinical information from users, (2) lab results, and (3) certain clinical protocols, such as, but not limited to, fertility health intake protocols 130, fertility treatment protocols 132, and/or fertility SOAP notes 134, to generate a treatment and prescribing plan and specific therapies for the user 105.

Further, fertility health system 100, fertility health application 110, and/or method 200 may be used to scrub (1) patient self-reported information against (2) lab results and against (3) certain clinical protocols (e.g., fertility health intake protocols 130, fertility treatment protocols 132, and/or fertility SOAP notes 134) to create a customized treatment plan for the user 105. Further, machine learning may be used to optimize the diagnosis and therapies provided to the user 105.

Further, in fertility health system 100, fertility health application 110, and/or method 200, machine learning may be used to optimize the diagnosis and therapies provided to the patient. For example, different types of assessment data may include, but is not limited to, layperson data, clinical data, biometric data, video data, medical data, heuristic date, and the like. Further, different types of assessment data may be weighted differently.

Further, the fertility health system 100, fertility health application 110, fertility health intake protocols 130, fertility treatment protocols 132, fertility SOAP notes 134, and method 200 as described herein is not limited to supporting fertility health only. The fertility health system 100, fertility health intake protocols 130, fertility treatment protocols 132, fertility SOAP notes 134, and method 200 may be suitable for guiding users and/or healthcare providers with respect to other health issues, such as, but not limited to, trimester-one pregnancy issues and/or postpartum issues. For example, see “1st trimester of pregnancy” portion of fertility treatment protocols 132 of fertility health application 110.

The fertility health system 100 and methods for fertility/reproductive health treatment solutions may be used, in one non-limiting example, by employers as a benefit and offered as a per eligible per month model, and may include additional charges for utilization. The fertility health system 100 and methods focuses on avoiding costly IVF interventions, which provides a unique offering in the fertility treatment and benefits marketplace, especially for employers looking to offer such benefits to their employees.

The fertility health system 100 and methods may be particularly targeted/attractive to health plans, pharmacy benefit managers (PBMs), employers, brokers, self-insured employers, third party administrators, captive providers, and other benefit providers as a benefit offering because of its process of providing fertility step-therapy treatment prior to the use of much more costly fertility interventions (e.g., IVF treatment).

For trimester one and postpartum support, the fertility health system 100 and methods provides the same model for treatment as it does for fertility health, for example, providing at-home labs (or walk in labs) with telehealth and prescription delivery .

The fertility health system 100 and methods can uniquely provide a nationwide fertility/reproductive health and treatment system (e.g., combining at-home labs, fertility specific telehealth, and prescription delivery).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A fertility/reproductive health system, comprising:
a. a server, the server including a fertility/reproductive health application, a controller, operating memory, and a communications interface, wherein the server is accessible via a network;
b. a data store in communication with the server; and
c. wherein the controller is configured to execute stored program instructions, comprising:
i. providing access to a user and one or more provider entities;
ii. receiving health data of a user;
iii. processing the received health data of the user;
iv. generating notifications to and/or from one or both of the user and the one or more provider entities; and
v. generating fertility/reproductive treatment suggestions based on the processed health data of the user.

2. The system of claim 1, wherein the received health data comprises one or more of data input by the user, responses to fertility/reproductive health intake protocols by the user, user's lab test results, and health data of the user received from one or more provider entities.

3. The system of claim 1, wherein the controller is further configured to execute stored program instructions, comprising sending one or more instructions to the user, based on at least some of the processed health data of the user, instructing the user to take certain lab tests at certain times.

4. The system of claim 2, wherein one or more of the lab tests include tests for one or more of anti-mullerian hormone (AMH), thyroid-stimulating hormone (TSH), follicle-stimulating hormone (FSH), estradiol (E2), luteinizing hormone (LH), progesterone (Pg), and total testosterone (TT), thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), free thyroxine (FT4), saliva testing, and/or semen analysis including testing for one or more of semen volume, concentration, count, motility, total motile count, morphology and/or DNA fragmentation.

5. The system of claim 2, wherein generating fertility/reproductive treatment suggestions comprises comparing the health data of the user to a set of fertility/reproductive treatment protocols.

6. The system of claim 1, wherein the one or more provider entities comprise one or more of a healthcare provider, laboratory service provider, pharmacy, and an in vitro fertilization (IVF) provider.

7. The system of claim 1, wherein the user accesses the fertility health application of the fertility health system through the network via a web-based portal via a computing device.

8. The system of claim 1, wherein the user accesses the fertility health application of the fertility health system via a local fertility health application installed on a computing device.

9. A method of using a fertility/reproductive health system, the method comprising:
a. accessing a fertility/reproductive health system by a user, the fertility/reproductive health system comprising:
i. a server, the server including a fertility/reproductive health application, a controller, operating memory, and a communications interface, wherein the server is accessible via a network;
ii. a data store in communication with the server; and
iii. wherein the controller is configured to execute stored program instructions, comprising:
1. providing access to a user and one or more provider entities;
2. receiving health data of a user;
3. processing the received health data of the user;
4. generating notifications to and/or from one or both of the user and the one or more provider entities; and
5. generating fertility/reproductive treatment suggestions based on the processed health data of the user;
b. entering health data of the user;
c. user taking lab tests as and when instructed by the fertility/reproductive health application;
d. entering lab test results into the fertility/reproductive health application; and
e. user consulting with a healthcare provider.

10. The method of claim 9, wherein the health data of the user comprises one or more of health data input directly by the user, responses to fertility/reproductive health intake protocols by the user, and health data of the user received from one or more provider entities.

11. The method of claim 9, wherein lab test results are entered by one or more of the user, laboratory service provider, and healthcare provider.

12. The method of claim 9, further comprising receiving fertility/reproductive treatment suggestions from the healthcare provider, wherein the fertility/reproductive treatment suggestions are generated based on comparing the entered health data of the user and lab test results of the user to a set of fertility/reproductive treatment protocols stored in the data store.

13. The method of claim 9, wherein the lab tests are either or both of at-home or at-lab lab tests.

14. The method of claim 9, wherein the user taking lab tests comprises the user, when instructed by the fertility/reproductive health application, taking one or more of a day-two menstrual cycle test, wherein the day-two menstrual cycle test comprises tests for one or more of anti-mullerian hormone (AMH), thyroid peroxidase antibody (TPoAB), free triiodothyronine (FT3), free thyroxine (FT4), fasting insulin (FI), hemoglobin A1C (HgbA1c), thyroid-stimulating hormone (TSH), dehydroepiandrosterone sulfate (DHEA-s), follicle-stimulating hormone (FSH), estradiol (E2), vitamin D (Vit D), luteinizing hormone (LH), saliva test, and/or semen analysis including testing for one or more of semen volume, concentration, count, motility, total motile count, morphology and/or DNA fragmentation.

15. The method of claim 14, wherein the user taking lab tests further comprises the user, after taking the day-two menstrual cycle test and/or when instructed by the fertility/reproductive health application, taking one or more ovulation tests, and wherein upon seven (7) days after a positive ovulation predictor and/or when instructed by the fertility/reproductive health application taking a progesterone (Pg) test and a testosterone (TT) test.

16. The method of claim 9, wherein the user taking lab tests comprises one or more of lab tests for one or more of semen analysis including semen volume, concentration, count, motility, total motile count, morphology and DNA fragmentation; and saliva testing.

17. Method of a using a fertility/reproductive health system, the method comprising:
a. providing a fertility/reproductive health system, comprising:
i. a server, the server including a fertility/reproductive health application, a controller, operating memory, and a communications interface, wherein the server is accessible via a network;
ii. a data store in communication with the server; and
iii. wherein the controller is configured to execute stored program instructions, comprising:
1. providing access to a user and one or more provider entities;
2. receiving health data of a user;
3. processing the received health data of the user;
4. generating notifications to and/or from one or both of the user and the one or more provider entities; and
5. generating fertility/reproductive treatment suggestions based on the processed health data of the user;
b. receiving and processing the health data of the user;
c. instructing the user when to conduct certain lab tests;
d. receiving and processing the lab test results of the user;
e. analyzing the processed health data and the processed lab test results in comparison to a set of fertility/reproductive treatment protocols stored in the data store; and
f. generating fertility/reproductive treatment suggestions based on the analyzed processed health data lab test results of the user in comparison to a set of fertility/reproductive treatment protocols stored in the data store.

18. The method of claim 17, further comprising presenting the generated fertility/reproductive treatment suggestions to a healthcare provider, and wherein the healthcare provider consults with the user on a recommended treatment plan.

19. The method of claim 17, wherein the health data of the user comprises one or more of health data input directly by the user, responses to fertility/reproductive health intake protocols by the user, and health data of the user received from one or more provider entities.

20. The method of claim 17, wherein lab test results are received from one or more of the user, laboratory service provider, and healthcare provider.

21. The method of claim 17, wherein the lab tests are either or both of at-home or at-lab lab tests.

22. The method of claim 17, further comprising, prior to instructing the user when to conduct certain lab tests, providing the user a fertility health system kit, wherein the fertility health system kit, comprises:
a. one or more blood spot cards;
b. one or more ovulation tests; and
c. one or more pregnancy tests.

23. The method of claim 17, wherein one or more of the lab test results may include test results for one or more of anti-mullerian hormone (AMH), thyroid-stimulating hormone (TSH), follicle-stimulating hormone (FSH), estradiol (E2), luteinizing hormone (LH), progesterone (Pg), and total testosterone (TT), thyroid peroxidase antibody (TPoAB), fasting insulin (FI), hemoglobin A1C (HgbA1c), dehydroepiandrosterone sulfate (DHEA-s), vitamin D (Vit D), free triiodothyronine (FT3), and free thyroxine (FT4).

24. A fertility/reproductive health system kit, comprising:
a. one or more blood spot cards;
b. one or more ovulation tests; and
c. one or more pregnancy tests.
